(19) United States
(12) Patent Application Publication
(10) Pub. No.: US 2010/0241172 A1
(43) Pub. Date: Sep. 23, 2010

(54) PEDICLE SCREW INCLUDING STATIONARY AND MOVABLE MEMBERS FOR FACILITATING THE SURGICAL CORRECTION OF SPINAL DEFORMITIES

(76) Inventors: Ashok Biyani, Sylvania, OH (US); Gary Durivage, Graytown, OH (US)

Correspondence Address: MACMILLAN SOBANSKI & TODD, LLC, ONE MARITIME PLAZA FIFTH FLOOR, 720 WATER STREET, TOLEDO, OH, 43604-1619, US

(21) Appl. No.: 12/729,629

(22) Filed: Mar. 23, 2010

Related U.S. Application Data
(60) Provisional application No. 61/162,631, filed on Mar. 23, 2009.

(51) Int. Cl. A61B 17/88; A61B 17/86

(52) U.S. Cl. 606/279; 606/305

(57) ABSTRACT

A pedicle screw for use in the treatment of a spinal deformity includes a head assembly and a fastener portion extending from the head assembly. The pedicle screw includes a head assembly and a fastener portion that extends from the head assembly. The fastener portion extends from the head assembly and is adapted to be secured to a vertebra of a spinal column. The head assembly includes a stationary member and a movable member. The movable member is supported on the stationary member for movement (such as pivoting movement) relative thereto between an opened position and a closed position. When the movable member is in the opened position, the head assembly is adapted to receive a longitudinal member therein for treatment of a spinal deformity. Thereafter, the movable member can be moved to the closed position to properly align the pedicle screw with the longitudinal member. A locking member and a retention member can then be used to maintain the movable member in the closed position.

### STATEMENTS REGARDING FEDERALLY SPONSORED RESEARCH OR DEVELOPMENT AND CROSS REFERENCE TO RELATED APPLICATIONS

This invention was not made with any government support. This application claims the benefit of U.S. Provisional Application No. 61/162,631 filed Mar. 23, 2009, the disclosure of which is incorporated herein by reference.

### BACKGROUND OF THE INVENTION

This invention relates in general to the field of orthopedic surgery and more specifically to the area of spinal surgery. In particular, this invention relates to an improved structure for a pedicle screw that is adapted to facilitate the installation of a spinal fixation device that can assist in the proper alignment of vertebrae in a spinal column.

Scoliosis is a medical condition in which a spinal column is abnormally curved from side to side. The abnormal curvature is often three dimensional in nature, including both a lateral component and a rotational component. In many patients having severe scoliosis, surgical treatment is required to correct these spinal deformities. Such surgical treatment may involve a procedure in which two or more vertebrae are anchored to one other by means of a spinal fixation device. The spinal fixation device is adapted to properly align the vertebrae of the spinal column and provide support thereto by reducing or eliminating undesirable relative vertebral movement.

Known spinal fixation devices typically include a plurality of pedicle screws that are initially inserted at predetermined locations and are secured to respective misaligned vertebrae of the spinal column. An elongated longitudinal member, such as for example an alignment rod, may then be inserted in a conventional open or a percutaneous manner into the patient. The pedicle screws are subsequently brought into alignment and secured to the longitudinal member so as to properly align the vertebrae. The longitudinal member generally extends along the posterior aspect of the spinal column for maintaining proper alignment and providing support to the spinal column.

A typical pedicle screw includes a head portion having a threaded shank portion extending therefrom. The head portion usually has a U-shaped yoke provided thereon for receiving the longitudinal member therein. A mechanism is also provided for retaining the longitudinal member within the U-shaped yoke on the head portion. The threaded shank portion is sized and shaped to engage a pedicle region of a vertebra to secure the pedicle screw thereto.

Although known pedicle screws have functioned satisfactorily, it has been found that in some instances, it can be difficult to properly align the plurality of pedicle screws for subsequent insertion and retention of the longitudinal member therein. Additionally, current pedicle screws do not have built-in derotation and, therefore, typically require the performance of simultaneous derotation maneuvers (such as by using bilateral screw extenders) to be performed. Thus, it would be desirable to provide an improved structure for a pedicle screw that has built-in derotation capabilities and, therefore, is adapted to facilitate the installation of the longitudinal member and, therefore, accomplish the proper alignment of the spinal column by means of the spinal fixation device.

### SUMMARY OF THE INVENTION

This invention relates to a pedicle screw for use in the treatment of a spinal deformity. The pedicle screw includes a head assembly and a fastener portion that extends from the head assembly. The fastener portion extends from the head assembly and is adapted to be secured to a vertebra of a spinal column. The head assembly includes a stationary member and a movable member. The movable member is supported on the stationary member for movement (such as pivoting movement) relative thereto between an opened position and a closed position. When the movable member is in the opened position, the head assembly is adapted to receive a longitudinal member therein for treatment of a spinal deformity. Thereafter, the movable member can be moved to the closed position to properly align the pedicle screw with the longitudinal member. A locking member and a retention member can then be used to maintain the movable member in the closed position.

Various objects and advantages will become apparent to those skilled in the art from the following detailed description of the preferred embodiments, when read in light of the accompanying drawings.

### BRIEF DESCRIPTION OF THE DRAWINGS

FIG. 1 is a schematic side elevational view of a spinal column having a spinal fixation device secured thereto by a plurality of pedicle screws in accordance with this invention.

FIG. 2 is an enlarged, exploded view of a first embodiment of one of the pedicle screws illustrated in FIG. 1.

FIG. 3 is a perspective view of a portion of the first embodiment of the pedicle screw illustrated in FIG. 2 wherein a pivot member is shown in an initial assembly position relative to a stationary member.

FIG. 4 is a perspective view of the portion of the first embodiment of the pedicle screw illustrated in FIG. 3 wherein the pivot member in shown an opened position relative to the stationary member.

FIG. 5 is a perspective view of the portion of the first embodiment of the pedicle screw illustrated in FIG. 4 wherein the pivot member is shown in a closed position relative to the stationary member.

FIG. 6 is a perspective view of the portion of the first embodiment of the pedicle screw illustrated in FIG. 5 wherein a locking member is shown installed on the pedicle screw and a retention member is shown prior to installation.

FIG. 7 is a side elevational view of a modified pivot member that can be used in a second embodiment of a pedicle screw in accordance with this invention.

FIG. 8 is a side elevational view of another modified pivot member that can be used in a third embodiment of a pedicle screw in accordance with this invention.

FIG. 9 is a side elevational view of yet another modified pivot member that can be used in a fourth embodiment of a pedicle screw in accordance with this invention.

FIG. 10 is a side elevational view of a further modified pivot member that can be used in a fifth embodiment of a pedicle screw in accordance with this invention.

FIG. 11 is an enlarged, exploded view of a sixth embodiment of a pedicle screw in accordance with this invention.

FIG. 12 is a perspective view of the portion of the sixth embodiment of the pedicle screw illustrated in FIG. 11 wherein the pivot member is shown in an opened position relative to the stationary member.

FIG. 13 is a perspective view of the portion of the sixth embodiment of the pedicle screw illustrated in FIG. 12 wherein the pivot member is shown in a closed position relative to the stationary member.

FIG. 14 is a perspective view of the portion of the sixth embodiment of the pedicle screw illustrated in FIG. 13 wherein a locking member is shown installed on the pedicle screw and a retention member is shown prior to installation.

FIG. 15 is a perspective view of a modified pivot member that can be used in a seventh embodiment of a pedicle screw in accordance with this invention.

FIG. 16 is a perspective view of another modified pivot member that can be used in an eighth embodiment of a pedicle screw in accordance with this invention.

FIG. 17 is a perspective view of a further modified stationary member that can be used in a ninth embodiment of a pedicle screw in accordance with this invention.

FIG. 18 is a perspective view of a tenth embodiment of a pedicle screw in accordance with this invention.

### DETAILED DESCRIPTION OF THE PREFERRED EMBODIMENTS

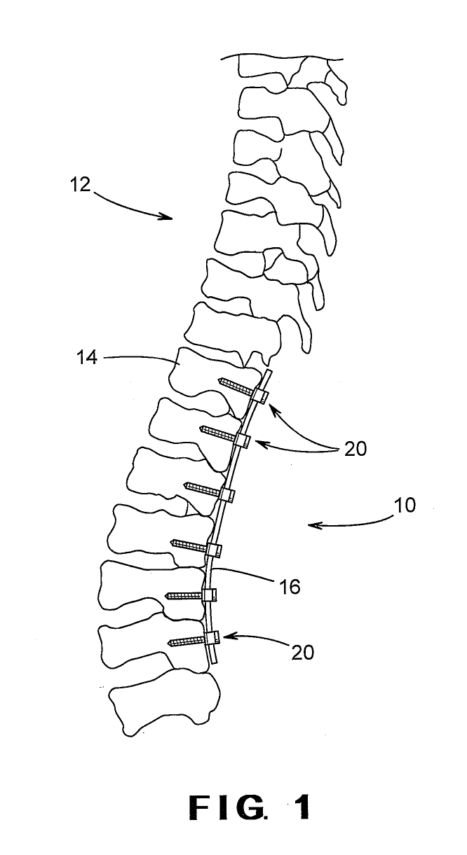

Referring now to the drawings, there is illustrated in FIG. 1 a spinal fixation device, indicated generally at 10, that is secured to a spinal column, indicated generally at 12. The spinal column 12 includes a plurality of vertebrae 14, and the spinal fixation device 10 can be secured to such vertebrae 14 in the manner described below to accomplish the surgical treatment of a spinal deformity. Although this invention will be described and illustrated in the context of treating pediatric or adult degenerative spinal deformities, it will be appreciated that this invention may be used to provide proper alignment and support of the spinal column 12 for any desired purpose. This invention may also be utilized for non-deformity cases, particularly in patients requiring long segment instrumentation during minimally invasive surgical procedures. In such cases, insertion of the pedicle screw of this invention may facilitate the placement of a conventional longitudinal member.

The spinal fixation device 10 can be used to properly align an abnormally curved portion of the spinal column 12 and to subsequently maintain the desired alignment thereof. To accomplish this, the spinal fixation device 10 may include a plurality of pedicle screws, each indicated generally at 20, and an elongated longitudinal member, such as for example an alignment rod 16, that extends between the pedicle screws 20. The pedicle screws 20 are respectively secured to a desired number of the vertebrae 14 of the spinal column 12 and are adapted to be secured to the alignment rod 16, as will be explained below. The alignment rod 16 may thus provide support for the spinal column 12 and maintain a desired alignment by reducing or eliminating an undesirable spinal deformity. As such, the alignment rod 16 may be pre-contoured to achieve a desired curvature and/or orientation of the spinal column 12. The pedicle screws 20 and the alignment rod 16 can be formed from any desired material or group of materials including, for example, stainless steel, titanium, cobalt chrome, and other materials that are suitable for use within a human body. Although not illustrated, the spinal fixation device 10 may incorporate additional instrumentation including, but not limited to, ratchet rods, lateral support rods, plates, wire members, or hooks if so desired without departing from the scope of the invention.

It will also be appreciated that spinal deformities may occur over any region or vertical length of the spinal column 12. Therefore, the spinal fixation device 10 may be positioned along any suitable surface of the spinal column 12 and may span any region or vertical length (i.e., any number of vertebrae 14) as desired. In the embodiment illustrated in FIG. 1, the spinal fixation device 10 is positioned along a posterior surface of the spinal column 12 and spans six adjacent vertebrae 14 of the lumbar and thoracic regions. However, the spinal fixation device 10 may be configured in any suitable manner for any desired application. Further, any suitable number of spinal fixation devices 10 may be incorporated without departing from the scope of the invention.

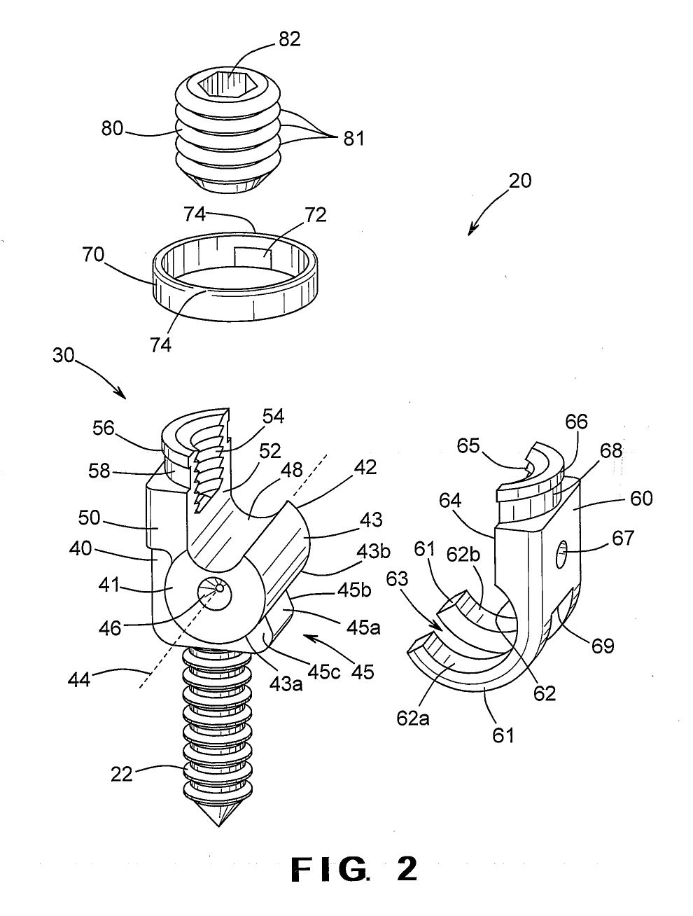

Referring now to FIG. 2, there is illustrated an exploded assembly view of a first embodiment of one of the pedicle screws 20. As shown therein, the pedicle screw 20 includes a head assembly, indicated generally at 30, having a fastener portion 22 extending therefrom. The head assembly 30 may include a stationary member 40, a movable or pivot member 60, a locking member 70, and a retention member 80. As will be explained in detail below, the stationary member 40 and the pivot member 60 of the head assembly 30 cooperate to define a generally U-shaped yoke for receiving and securing the alignment rod 16 to the pedicle screw 20. The head assembly 30 may thus facilitate proper alignment of the spinal column 12 and assist in installation of the spinal fixation device 10.

The head assembly 30 is formed integrally with or connected to the fastener portion 22 for mounting or otherwise securing the head assembly 30 to one of the vertebrae 14. In the illustrated embodiment, the fastener portion 22 is an elongated member having an externally threaded surface that is adapted to be inserted into a pedicle region of the vertebra 14. The fastener portion 22 can have any desired root diameter, thread diameter, pitch, and number of threads. If desired, the fastener portion 22 can be cannulated to allow it to be inserted over a guide wire (not shown), such as is used during minimally invasive surgical procedures. It should be fully understood, however, that the fastener portion 22 can be embodied as any structure that is configured to penetrate and/or engage the vertebra 14 so as to fix the head assembly 30 in position relative thereto.

The head assembly 30 can be connected to the fastener portion 22 by means of the stationary member 40. As illustrated, the stationary member 40 can be rigidly connected to the fastener portion 22 in a mono-axial fashion, i.e., fixed in position relative to one another. For example, the stationary member 40 and the fastener portion 22 may be formed from a single piece of material using any suitable process including, but not limited to, casting, molding, and/or machining. Alternatively, the stationary member 40 and the fastener portion 22 may be formed as separate components that are subsequently secured to one another in any suitable manner. For example, the fastener portion 22 may be a stanchion rod (not shown) initially anchored to the vertebra 14. Subsequently, the stationary member 40 may be connected or otherwise secured to the stanchion rod (not shown) thereby forming the pedicle screw 20. Further, it should be fully appreciated that the head assembly 30 may be pivotally connected to the fastener portion 22 in either a uni-axial or poly-axial fashion without departing from the scope of the invention.

The illustrated stationary member 40 has a first end surface 41 and a second end surface 42. In the illustrated embodiment, the first and second end surfaces 41 and 42 are generally flat and parallel surfaces that define opposed external end walls of the stationary member 40. However, it will be appreciated that the first and second end surfaces 41 and 42 may have any desired shape or combination of shapes and may be oriented as desired relative to one another. As used herein, a longitudinal length of the head assembly 30 is defined as the distance between the first and second end surfaces 41 and 42. The longitudinal length of the head assembly 30 is configured to extend along a portion of the alignment rod 16 when the alignment rod 16 is secured within the head assembly 30. It should be fully appreciated that the head assembly 30 may have any longitudinal length as desired for any intended application.

As described above, the stationary member 40 and the pivot member 60 of the head assembly 30 cooperate to define a generally U-shaped yoke for receiving and securing the alignment rod 16 therein. To accomplish this, the stationary member 40 can include a rod locating surface 48 that is configured to receive and locate the alignment rod 16 on and within the head assembly 30. In the illustrated embodiment, the rod locating surface 48 is a partially cylindrical surface that preferably corresponds at least generally with the external cylindrical surface of the illustrated alignment rod 16. The rod locating surface 48 can be provided on any surface of the stationary member 40 and may span the entire longitudinal length thereof. However, the rod locating surface 48 is preferably contoured so as to receive and locate the alignment rod 16 within the head assembly 30. Alternatively, the rod locating surface 48 may be a V-shaped or a notch-shaped surface. The rod locating surface 48 may, if desired, house a drive mechanism of any desired configuration for installation into the vertebrae 14. The rod locating surface 48 may also be cannulated and house a polyaxial screw head.

The stationary member 40 may include a distal portion 50 that defines a first portion of the generally U-shaped yoke portion of the head assembly 30. In the illustrated embodiment, the distal portion 50 includes a first lateral surface 52 that is located directly adjacent to and extends upwardly from the rod locating surface 48. The first lateral surface 52 may be configured to locate and secure the alignment rod 16 within the U-shaped yoke portion of the head assembly 30, as will be explained below. Thus, the first lateral surface 52 can be a generally flat surface that spans the longitudinal length of the stationary member 40. However, the first lateral surface 52 may be formed as any desired surface that is adapted to locate and secure the alignment rod 16 to the head assembly 30, such as for example a V-shaped or concave-shaped surface. Further, the first lateral surface 52 may be positioned along or span any longitudinal length of the distal portion 50 if so desired.

The illustrated distal portion 50 of the stationary member 40 also includes one or more internal mounting threads 54 that are provided to secure the externally threaded retention member 80 to the head assembly 30. As will be explained below, the internal mounting threads 54 correspond with a corresponding internal mounting threads 65 that are provided on the pivot member 60 to facilitate the securement the retention member 80 therebetween. In the illustrated embodiment, the internal mounting threads 54 of the distal portion 50 form a semi-annular, internally threaded section. The internal mounting threads 54 may be provided in the first lateral surface 52 so as to form a depression therein. The internal mounting threads 54 can have any desired root diameter, thread diameter, pitch, and number of threads. The first lateral surface 52 may alternatively be adapted to accept a conventional non-threaded locking mechanism (not shown).

The illustrated distal portion 50 of the stationary member 40 further includes a locking lip 56 that is located at an end portion thereof for facilitating the securement of the pivot member 60 to the stationary member 40, as will be explained below. In the illustrated embodiment, the locking lip 56 is defined by an external surface of the distal portion 50 that extends circumferentially from an end portion of the first lateral surface 52. The locking lip 56 may be formed in the shape of a generally semi-annular structure that encompasses the internal mounting threads 54 described above. The locking lip 56 may, if desired, have a chamfered outer and/or leading edge (not shown) to aid in locating the locking member 70 thereto. It will be understood, however, that the locking lip 56 may be configured or shaped in any suitable manner so as to perform the functions described herein or, alternatively, may be omitted entirely.

The distal portion 50 of the stationary member 40 may additionally have a locking groove 58 provided therein to facilitate the securement of the locking member 70 to the head assembly 30, as will be explained below. In the illustrated embodiment, the locking groove 58 is a channel or similar undercut portion provided in an external surface of the distal portion 50 that is located axially adjacent to the locking lip 56. The locking groove 58 may define a semi-annular structure that circumferentially extends from the end portions of the first lateral surface 52 so as to encompass the internal mounting threads 54. However, the locking groove 58 may be configured or shaped in any suitable manner so as to perform the functions described herein. For example, the locking groove 58 may include one or more external mounting threads (not shown) if desired. The locking groove 58 may also be configured to accept a unitized “innie” 80 and “outie” 70.

The pivot member 60 supported for pivoting or other movement relative to the stationary member 40. To accomplish this, the stationary member 40 may have a pivot engagement surface 43 provided thereon. In the illustrated embodiment, the pivot engagement surface 43 is a partially cylindrical surface that extends between and is generally perpendicular to the first and second end surfaces 41 and 42. The pivot engagement surface 43 can be an external surface of the stationary member 40 that intersects with the rod locating surface 48. However, the pivot engagement surface 43 can be any desired shape or combination of shapes so as to perform the functions described herein. Furthermore, the pivot engagement surface 43 may include any number or combinations of engagement surfaces spaced along the longitudinal length of the stationary member 40, as will be explained below.

The illustrated pivot engagement surface 43 defines a pivot axis 44. The illustrated pivot axis 44 spans the longitudinal length of the stationary member 40 as described above. In the illustrated embodiment, the pivot axis 44 preferably intersects with and extends between the first and second end surfaces 41 and 42. As a result, the pivot axis 44 extends through a central portion of the stationary member 40. However, it should be fully appreciated that the pivot axis 44 may be offset relative to the central portion of the stationary member 40 if so desired.

The stationary member 40 may also include a pivot stop, indicated generally at 45, that is adapted to prevent the pivot member 60 from moving beyond a predetermined opened position relative to the stationary member 40. The pivot stop 45 may also facilitate the alignment of the pivot member 60 on the stationary member 40, as will be explained below. In the illustrated embodiment, the pivot stop 45 includes a pivot stop surface 45a that projects from the pivot engagement surface 43 along the longitudinal length thereof. The pivot stop 45 may also include opposed aligning surfaces 45b and 45c that project from the pivot engagement surface 43 and extend generally along a circumference thereof. It will be appreciated, however, that the pivot stop 45 can be arranged in any desirable manner so as to perform the functions described herein.

As illustrated in FIG. 2, the stationary member 40 may include one or more pivot engagement surfaces, such as shown at 43a and 43b, that are spaced apart from one another along the longitudinal length of the stationary member 40. In the illustrated embodiment, the two pivot engagement surfaces 43a and 43b extend generally circumferentially adjacent to and along the opposite aligning surfaces 45b and 45c of the pivot stop 45. The illustrated pivot engagement surfaces 43a and 43b define a substantially cylindrical and continuous pivot engagement surface 43 that extends about the pivot axis 44, the purpose of which will be explained below. However, as described above, the stationary member 40 can include any number or combination of multiple pivot engagement surfaces so as to perform the functions described herein.

The stationary member 40 may include one or more installation tool locators 46 that are adapted to facilitate the attachment or otherwise secure a tool (not shown) to the head assembly 30 for installation and/or manipulation of the pedicle screw 20. For example, the installation tool locator 46 can be an indentation or cavity configured to receive desired tooling for this purpose. However, the installation tool locator 46 may be embodied in any suitable manner for securing the tool to the head assembly 30. In the illustrated embodiment, two installation tool locators 46 (only one is illustrated) are provided on the opposite first and second end surfaces 41 and 42 of the stationary member 40. The installation tool locators 46 are preferably positioned along the pivot axis 44 defined by the pivot engagement surface 43. It should be appreciated, however, that the installation tool locators 46 may positioned along any portion or external surfaces of the head assembly 30.

As shown in FIG. 2, the illustrated pivot member 60 includes a pair of mounting members 61, although any number of such mounting members 61 may be provided as desired. The mounting members 61 are provided to support the pivot member 60 on the stationary member 40 for movement relative thereto. In the illustrated embodiment, the mounting members 61 are semi-annular in shape so as to extend about the pivot axis 44. As will be described below, the mounting members 61 may correspond in number and shape with the multiple pivot engagement surfaces 43a and 43b of the stationary member 40. If desired, the mounting members 61 can extend more than one hundred eighty degrees about the pivot axis 44 so as to permanently support the pivot member 60 on the stationary member 40. Alternatively, as shown in the illustrated embodiment, the mounting members 61 may extend less than one hundred eighty degrees about the pivot axis 44 so as to removably support the pivot member 60 to the stationary member 40. The mounting members 61 may have any desired shape or combination of shapes so as to perform the functions described herein. Furthermore, the pivot member 60 may be pivotally mounted to the stationary member 40 in any suitable manner without departing from the scope of this invention.

The mounting members 61 may be spaced apart so as to define a clearance or space, indicated generally at 63, therebetween. The clearance 63 can be configured to locate and maintain a desired alignment of the pivot member 60 relative to the stationary member 40. For example, the mounting members 61 may abut the opposite side surfaces 45b and 45c of the pivot stop 45. This configuration prevents undesirable movement of the pivot member 60 in a longitudinal direction along the pivot axis 44. However, the pivot member 60 may maintain proper alignment to the stationary member 40 in any suitable manner.

The pivot member 60, including the two mounting members 61, may define a pivot engagement surface 62. In the illustrated embodiment, the pivot engagement surface 62 is a generally cylindrical surface of the pivot member 60. The pivot engagement surface 62 may be contoured to correspond with the pivot engagement surface 43 provided on the stationary member 40 for sliding engagement therewith. It will be appreciated that the pivot engagement surface 62 may span any desired longitudinal length of the pivot member 60. Furthermore, the pivot member 60 may include any number of engagement surfaces separately spaced along the longitudinal length of the pivot member 60. For example, the pivot engagement surface 62 may include multiple pivot engagement surfaces 62a and 62b that are respectively provided on surfaces of the two mounting members 61. As illustrated, the multiple pivot engagement surfaces 62a and 62b can be contoured to correspond with the multiple pivot engagement surfaces 43a and 43b provided on the stationary member 40 for sliding engagement therewith. However, the pivot engagement surface 62 may be contoured in any manner so as to perform the functions described herein.

The pivot member 60 may also define a pivot stop surface 69 that is configured to prevent pivotal movement of the pivot member 60 beyond a predetermined opened position relative to the stationary member 40. In the illustrated embodiment, the pivot stop surface 69 is a generally flat surface adapted to selectively contact the pivot stop surface 45a provided on the stationary member 40. The pivot stop surface 69 can partially define an end portion of the clearance 63 spanning the two mounting members 61. However, the pivot stop surface 69 can be contoured in any suitable manner and be located along any portion of the pivot member 60 so as to perform the functions described herein. The pivot engagement surfaces 62 and 43, respectively, of the pivot member 60 and the stationary member 40 may have relatively smooth gliding surfaces provided thereon. Alternatively, the pivot engagement surfaces 62 and 43, respectively, of the pivot member 60 and the stationary member 40 may have relatively rough surfaces provided thereon to provide some frictional resistance to relative movement and, as a result, decrease looseness or floppiness therebetween.

The pivot member 60 may also include a distal portion defining a second lateral surface 64. The second lateral surface 64 is configured to cooperate with the first lateral surface 52 so as to form the generally U-shaped yoke configuration for the head assembly 30 described above. As will be explained below, the second lateral surface 64 can be pivoted or otherwise moved relative to the first lateral surface 52 so as to initially receive and then subsequently retain the alignment rod 16 within the U-shaped yoke portion of the head assembly 30. In the illustrated embodiment, the second lateral surface 64 can be a generally flat surface extending along the longitudinal length of the pivot member 60. However, the second lateral surface 64 may be contoured in any suitable manner to secure the alignment rod 16 within the head assembly 30, such as for example a V-shaped or concave shaped surface. Furthermore, the second lateral surface 64 may be positioned or span any longitudinal length of the pivot member 60 as desired.

The distal portion of the pivot member 60 may have internal mounting threads 65 formed therein that are adapted to secure the externally threaded retention member 80 to the head assembly 30. As described above, the internal mounting threads 65 may correspond in size and shape with the internal mounting threads 54 provided on the stationary member 40 to secure the retention member 80 to the head assembly 30. As a result, the retention member 80 may be secured to the head assembly 30 for selective engagement with the alignment rod 16, as will be further explained below. In the illustrated embodiment, the internal mounting threads 65 form a semi-annular, internally threaded section. The internal mounting threads 65 may be located along the second lateral surface 64 so as to form a depression therein. Furthermore, the internal mounting threads 65 can have any desired root diameter, thread diameter, pitch, and number of threads. The second lateral surface 64 of the pivot member 60 may also be adapted to accept conventional non-threaded locking mechanism that are known in the art.

The distal portion of the pivot member 60 may also have a locking lip 66 that is located at an end portion thereof for securing the pivot member 60 to the stationary member 40, as will be explained below. In the illustrated embodiment, the locking lip 66 is defined by an external surface of the pivot member 60 that circumferentially extends from end portions of the second lateral surface 64. The locking lip 66 may form a semi-annular structure so as to encompass the internal mounting threads 65 described above. Furthermore, the locking lip 66 may include a chamfered outer edge (not shown) to aid in locating the locking member 70 thereto. It will be understood, however, that the locking lip 66 may be configured or shaped in any suitable manner so as to perform the functions described herein.

The distal portion of the pivot member 60 may further have a locking groove 68 formed therein for securing the locking member 70 to the head assembly 30, as will be explained below. In the illustrated embodiment, the locking groove 68 is a channel or undercut in an external surface of the pivot member 60 that is located axially adjacent to the locking lip 66. The locking groove 68 may define a semi-annular structure that circumferentially extends from end portions of the second lateral surface 64 so as to encompass the internal mounting threads 65. However, the locking groove 68 may be configured or shaped in any suitable manner so as to perform the functions described herein. For example, the locking groove 68 may include external mounting threads (not shown) if so desired.

Lastly, the pivot member 60 may include a pivot tool locator 67, although such is not required. The pivot tool locator 67 can be provided for locating and securing desired a tool to the pivot member 60 to aid in pivotal movement of the pivot member 60. As such, the tool locator 67 may define a contoured surface, such as for example an indentation or protrusion, and can be located on any surface of the pivot member 60. It will be appreciated that the tool locator 67 may be embodied as any structure configured to aid in pivotal movement of the pivot member 60.

As mentioned above, the head assembly 30 may include a locking member 70. The locking member 70 is provided to retain the pivot member 60 to the stationary member 40 in the closed position for securing the alignment rod 16 therebetween. In the illustrated embodiment, the locking member 70 is generally annular in shape, defining an internal diameter. The internal diameter of the illustrated locking member 70 is adapted to encompass and engage the locking lip 56 of the stationary member 40 and the locking lip 66 of the pivot member 60. In doing so, the locking member 70 secures the pivot member 60 relative to the stationary member 40. However, the locking member 70 may be configured in any suitable manner for locking the pivot member 60 to the stationary member 40. For example, the locking member 70 may be a latch or cap style component if so desired. The locking member 70 may also include a built-in friction lock or similar mechanism to prevent unintentional unwinding and loosening.

The locking member 70 may include one or more locking tabs 72 that are adapted to secure the locking member 70 to the head assembly 30. In the illustrated embodiment, a single locking tab 72 protrudes radially inwardly from the internal diameter of the locking member 70. The locking tab 72 may be shaped to correspond with the locking grooves 58 and 68 such that the locking tab 72 may be received therein, as will be explained below. As embodied, the locking member 70 may include any desired number of locking tabs 72, and the locking tab(s) 72 may be configured in any suitable manner for securing the locking member 70 to the head assembly 30. For example, the locking member 70 may have an internally threaded diameter (not shown).

The locking member 70 may also include one or more position indicators 74 that are provided to indicate the rotational position of the locking member 70 relative to remainder of the head assembly 30. In the illustrated embodiment, the position indicator 74 is a pair of marks provided on the external portion of the locking member 70 that indicate the rotational location of the locking tab 72. The position indicator 74 may, however, be any visual or physical feature located on any portion of the locking member 70 so as to perform the functions described herein, including non-threaded retention mechanisms.

The locking member 70 may further include a rotational tool locator (not shown) to assist in rotational movement of the locking member 70. For example, the rotational tool locator may be a cavity or protrusion located on an external surface of the locking member 70. The rotational tool locator can be adapted for engaging or otherwise securing tooling thereto so as to rotate the locking member 70. As such, it should be understood that the rotational tool locator may be any structure that is adapted to aid in rotation of the locking member 70.

As briefly described above, the head assembly 30 includes a retention member 80 that is adapted to retain or otherwise secure the alignment rod 16 between the first lateral surface 52 and the second lateral surface 64. In the illustrated embodiment, the retention member 80 is an elongated component having an externally threaded portion 81 for engaging the internal mounting threads 54 and 65. The internal mounting threads 54 and 65 are configured to support the retention member 80 for linear movement in a generally perpendicular direction relative to the alignment rod 16 when the retention member 80 is rotated relative thereto, as will be explained below. In the illustrated embodiment, the retention member 80 is externally threaded along its entire length, thus permitting the retention member 80 to be fully disposed within the head assembly 30. However, the retention member 80 may be configured in any suitable manner to accomplish the purposes described herein.

As illustrated, the retention member 80 may have a structure provided thereon that is adapted to be engaged by a tool (not shown) or similar device to effect rotation of the retention member 80 relative to the head portion 30. In the illustrated embodiment, an end portion of the retention member 80 has an internal key way 82 formed therein for receiving such a tool, such as for example a hexagonal wrench. As briefly described above, rotation of the retention member 80 causes linear movement thereof in a generally perpendicular direction relative to the alignment rod 16. The retention member 80 may, however, be embodied in any suitable manner to accomplish the purposes described herein, including but not limited to a head style configuration (not shown).

It will be appreciated that the head assembly 30 may include any number of pivot members 60 that are configured to secure the alignment rod 16 therebetween. For example, in addition to the pivot member 60 being pivotally supported on the stationary member 40, the distal portion 50 of the stationary member 40 may also be pivotally mounted thereto.

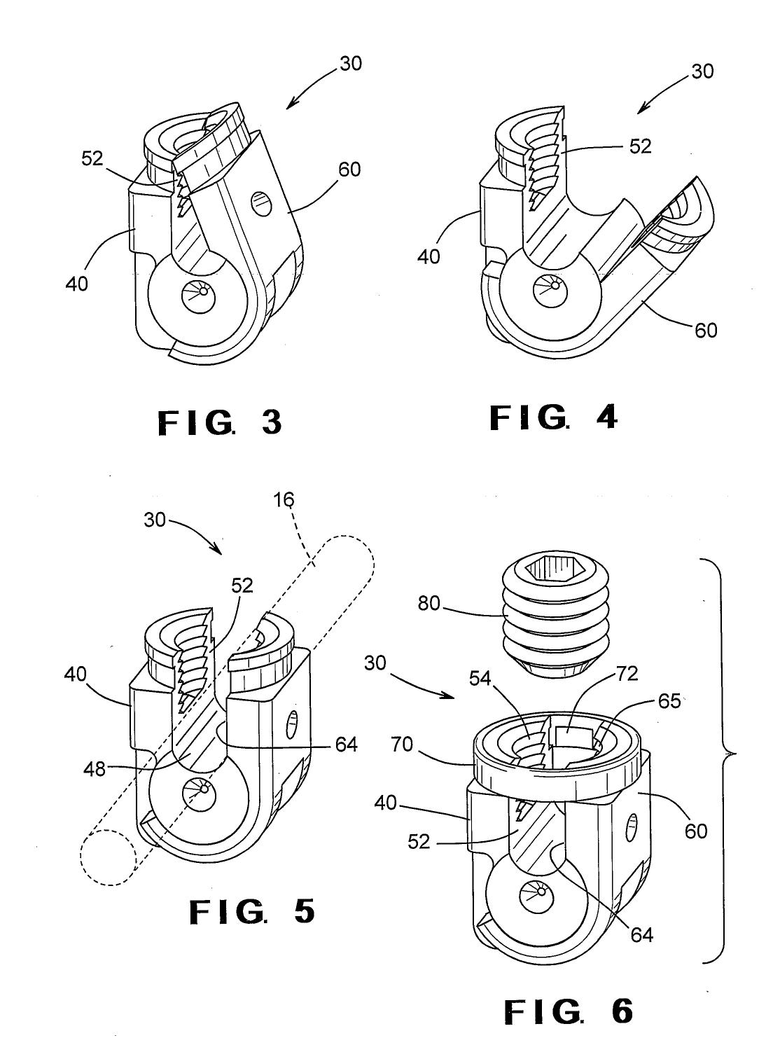

The installation and assembly of the spinal fixation device 10 will now be described. The stationary member 40 and the pivot member 60 are initially oriented as generally shown in FIG. 3 prior to being assembled together. The leading surfaces of the pivot engagement surfaces 62a and 62b of the pivot member 30 are, in this initial assembly position, located at their lowest points (closest to the fastener portion 22) and, thus, clear the pivot engagement surfaces 43a and 43b of the stationary member 40. This relationship allows clearance of the mounting members 61 and the pivot engagement surface 62 and facilitates engagement of the mating surfaces. The stationary member 40 and the pivot member 60 are then moved into engagement with one another as shown in FIG. 3. The stationary member 40 and the pivot member 60 of the pedicle screw 20 can be preassembled in this manner during the manufacturing process. If desired, the pivot member 60 can fit snugly or be otherwise affirmatively retained on the stationary member 40 by a blocking structure (not shown) provided on either or both of the stationary member 40 and the pivot member 60 such that the initial assembly position illustrated in FIG. 3 is unattainable after the assembly of the stationary member 40 and the pivot member 60 during the manufacturing process. Alternatively, a separate pin, detent, or similar blocking mechanism (not shown) may be installed to prevent the stationary member 40 and the pivot member 60 from moving back into the initial assembly position illustrated in FIG. 3 after assembly. The use of the blocking structure or the blocking mechanism would insure that once assembled together, the stationary member 40 and the pivot member 60 cannot become separated later, such as during a surgical procedure.

During the surgical procedure, any desired number of the pedicle screws 20 can be secured to respective vertebrae 14 of the spinal column 12. To accomplish this, the fastener portions 22 of the pedicle screws 20 can be inserted into respective pedicle regions of the vertebrae 14. Each pedicle screw 20 may be installed using any desired procedure and installation devices. For example, special installation tooling (not shown), such as an extension tube, may be secured to the installation tool locators 46 of the stationary member 40 for facilitating the insertion and positioning the pedicle screws 20.

Once the pedicle screws 20 have been secured to the respective vertebrae 14, the pivot members 60 are moved to the opened positions relative to stationary members 40 as shown in FIG. 4, wherein the second lateral surfaces 64 of the pivot members 60 are moved away from the first lateral surfaces 52 of the stationary members 40. Such orientation of the pivot members 60 can facilitate the process of installing the alignment rod 16. In the opened position, the second lateral surface 64 of the pivot member 60 is spaced apart from the first lateral surface 52 of the stationary member 40. The opened position can be achieved by pivoting or otherwise moving the pivot member 60 relative to the stationary member 40 as described above until the pivot stop surface 69 of the pivot member engages the associated pivot stop 45 of the stationary member 40. this occurs when the pivot member 60 is in the opened position relative to the stationary member 40, as shown in FIG. 4. Such engagement prevents further pivoting movement of the pivot member 60 relative to the stationary member 40. By pivotally moving the pivot member 60 away from the stationary member 40, the second lateral surface 64 is moved relatively away from the first lateral surface 52, thereby splaying open the head assembly 30. In the illustrated embodiment, the pivot member 60 pivots approximately 45° away from the stationary member 40 to reach the opened position. However, the head assembly 30 may be configured to permit any desired amount of pivoting movement. Such orientation of the pivot members 60 facilitates the process of installing the alignment rod 16 by providing a relative wide space for the alignment rod 16 to be received within the head portion 30 of the pedicle screw 20, as opposed to conventional pedicles screws in which the first and second lateral surfaces 52 and 64 are fixed in position relative to one another.

The pedicle screws 20 can then be manually or otherwise aligned so as to facilitate the positioning of the alignment rod 16 within the splayed open head assemblies 30. The alignment rod 16 can be inserted using any desired procedure and installation devices. For example, the alignment rod 16 may be percutaneously inserted into the patient using any minimally invasive procedure or by utilizing conventional open techniques. After the alignment rod 16 has been disposed between the first and second lateral surfaces 52 and 64 of the pivot member 60 and the stationary member 40, the pivot member 60 is pivoted or otherwise moved from the opened position relative to the stationary member 40 to the closed position shown in FIG. 5. The pivot member 60 can be pivoted toward the stationary member 40 until the head assembly 30 is properly aligned with the alignment rod 16. Proper alignment is achieved when the alignment rod 16 is fully seated against the rod locating surface 48 and is fully engaged on both sides by the first and second lateral surfaces 52 and 64. Once the alignment rod 16 has been so seated, the stationary member 30 and the pivot member 60 cannot return to the initial assembly position illustrated in FIG. 3 and, therefore, cannot become inadvertently disengaged from one another.

Referring now to FIG. 6, once the pivot member 60 has been moved to the closed position relative to stationary member 40, the locking member 70 may be positioned over the distal portions of the stationary member 40 and the pivot member 60. As described above, the locking member 70 can be configured to secure the pivot member 60 and the stationary member 40 together in this closed position. In the illustrated embodiment, the locking member 70 is initially oriented such that the locking tab 72 is disposed within the U-shaped yoke defined between the first lateral surface 52 and the second lateral surface 64. The locking member 70 can then be rotated so as to position the locking tab 72 within the locking groove 74 for securing the locking member 70 to the head assembly 30.

The retention member 80 may then be inserted through the locking member 70 so as to threadably engage the internal mounting threads 54 and 65 respectively provided on the stationary member 40 and the pivot member 60. As described above, the internal mounting threads 54 and 65 may be configured to engaged the externally threaded portion 81 of the retention member 80 for linear movement relative to the alignment rod 16 when the retention member 80 is rotated. As a result, an end portion of the retention member 80 can be moved into direct engagement with an external surface of the alignment rod 16 for firmly securing the alignment rod 16 within the head assembly 30.

The methods described above and illustrated in FIGS. 3 through 6 may be repeated for each of the pedicle screws 20 positioned along the spinal column 12. As a result, the spinal fixation device 10 is capable of providing lateral and rotational support to the spinal column 12 so as to maintain proper alignment thereof.

Once fully assembled, the head assembly 30 may acquire any suitable shape for use within the human body. In the illustrated embodiment, the head assembly 30 is a generally cubical shaped component having a slender profile with rounded corners and chamfered edges. However, alternative shapes may be incorporated if so desired, such as for example by altering the pivot member 60 as will be explained below. Furthermore, it should be fully appreciated that any portion of the head assembly 30 may be specifically configured for an intended application.

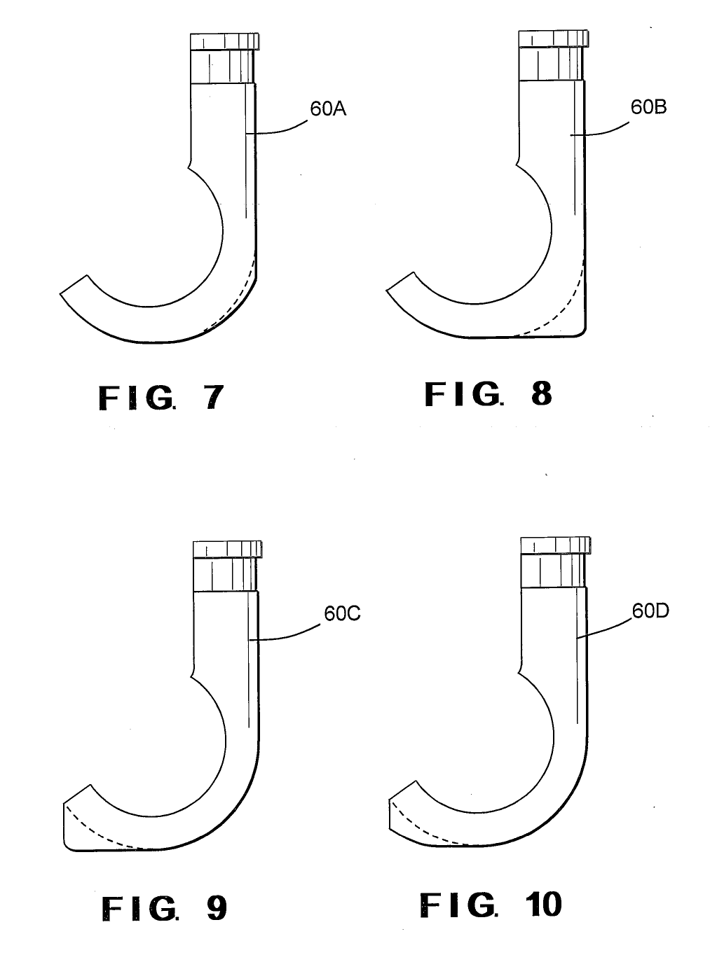

FIG. 7 is a side elevational view of a modified pivot member 60A that can be used in a second embodiment of a pedicle screw in accordance with this invention. The modified pivot member 60A has an increased lower anterior body thickness with a radii blend relative to the above-described pivot member 60, which is shown by a dotted line. The modified pivot member 60A is otherwise identical in structure and operation to the above-described pivot member 60.

FIG. 8 is a side elevational view of another modified pivot member 60B that can be used in a third embodiment of a pedicle screw in accordance with this invention. The modified pivot member 60B has an increased lower anterior body thickness with right angled flat surfaces on the mounting members 61 adjacent to the pivot engagement surface 62, as compared with the above-described pivot member 60 (which is shown by a dotted line). The modified pivot member 60B is otherwise identical in structure and operation to the above-described pivot member 60.

FIG. 9 is a side elevational view of yet another modified pivot member 60C that can be used in a fourth embodiment of a pedicle screw in accordance with this invention. The modified pivot member 60C has an increased lower anterior body thickness with right angled flat surfaces on the mounting members 61 remote from the pivot engagement surface 62, as compared with the above-described pivot member 60 (which is shown by a dotted line). The modified pivot member 60C is otherwise identical in structure and operation to the above-described pivot member 60. If desired, the lower surfaces of the mounting members 61 that come into contact with the vertebra 14 may have serrations or spikes (not shown) provided thereon to further improve the grasp of the pivot member 60 on the bone of the vertebra 14, which can help facilitate the derotation of the vertebra 14 as the two members 40 and 60 are brought from the opened position to the closed position.

FIG. 10 is a side elevational view of a further modified pivot member 60D that can be used in a fifth embodiment of a pedicle screw in accordance with this invention. The modified pivot member 60D has an increased lower anterior body thickness with obtusely angled flat surfaces on the mounting members 61 remote from the pivot engagement surface 62, as compared with the above-described pivot member 60 (which is shown by a dotted line). The modified pivot member 60D is otherwise identical in structure and operation to the above-described pivot member 60.

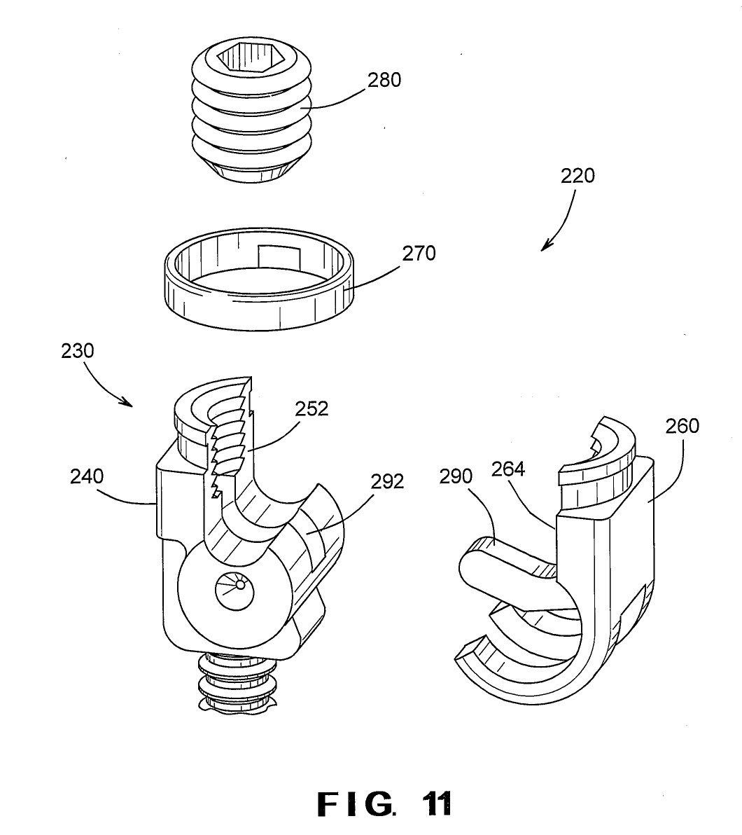

FIG. 11 is an enlarged, exploded view of a portion of a sixth embodiment of a pedicle screw, indicated generally at 220 illustrated in FIG. 1. The sixth embodiment of the pedicle screw 220 is, in large measure, similar to the first embodiment of the pedicle screw 20 described above, and like reference numbers (incremented by two hundred) are used to designate similar structures. The pivot member 260 of the sixth embodiment of the pedicle screw 220, however, has a pivot fulcrum 290 provided thereon. The pivot fulcrum 290 is adapted to automatically move the pivot member 260 from the opened position to the closed position, as will be explained below. In the illustrated embodiment, the pivot fulcrum 290 is an elongated member that extends generally laterally away from the pivot member 260 toward the stationary member 240. The pivot fulcrum 290 may extend from the pivot engagement surface 262 and be located directly adjacent the second lateral surface 264. In the illustrated embodiment, the pivot fulcrum 290 is centrally located along the longitudinal length of the pivot member 260. However, the pivot fulcrum 290 may be configured in any suitable manner or be provided at any location along the pivot member 260 so as to accomplish the functions described herein.

The stationary member 240 of the sixth embodiment of the pedicle screw 220 has a fulcrum pocket 292 provided therein that is adapted to correspond with and receive the pivot fulcrum 292 in the manner described below. In the illustrated embodiment, the fulcrum pocket 292 is a laterally extending recess formed in the stationary member 240. The fulcrum pocket 292 is located in alignment with the pivot fulcrum 290 provided on the pivot member 2670. However, the fulcrum pocket 292 may be configured in any suitable manner or be located along any portion of the stationary member 240 so as to accomplish the functions described herein.

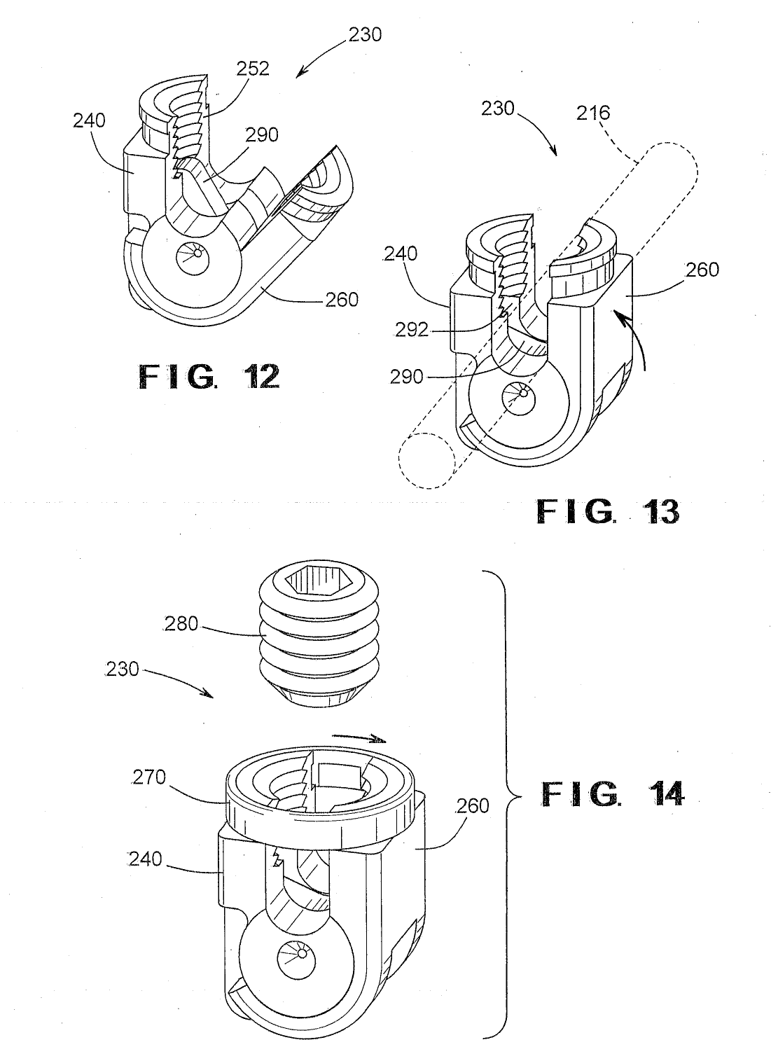

Referring now to FIGS. 12, 13, and 14, the operation of the head assembly 230 of the sixth embodiment of the pedicle screw 220 will now be described. Other than as explained herein, the methods for installing and assembling the pedicle screw 220 and the head assembly 230 are as described above in the first embodiment. As illustrated in FIG. 12, when the head assembly 230 is in the opened position, the pivot fulcrum 292 protrudes from the fulcrum pocket 294 and extends outwardly between the first and second lateral surfaces 252 and 264. The pedicle screw 220 can then be initially aligned so as to position an alignment rod 216 within the splayed open head assembly 230.

Referring now to FIG. 13, the pedicle screw 220 may be derotated by engagement of the alignment rod 216 with the pivot fulcrum 290. In the illustrated embodiment, forcible engagement of the alignment rod 216 by a threaded or non-threaded reduction mechanism with the pivot fulcrum 290 pivotally moves the pivot member 260 toward the stationary member 240. The pivot member 260 is pivoted toward the stationary member 240 until the head assembly 230 is properly aligned with the alignment rod 216, as described above. When properly aligned, the pivot fulcrum 290 is fully disposed within the fulcrum pocket 292 as illustrated. As should be appreciated, the head assembly 230 is now in the closed position.

Referring now to FIG. 14, once the head assembly 230 is in the closed position, the locking member 270 can be positioned over the head assembly 230 and secured thereto as describe above in the first embodiment. Similarly, the retention member 280 may then be inserted through the locking member 270 and secured to the head assembly 230 as described above in the first embodiment. An end portion of the retention member 280 can be moved into direct engagement with an external surface of the alignment rod 216 for firmly securing the alignment rod 216 within the head assembly 230.

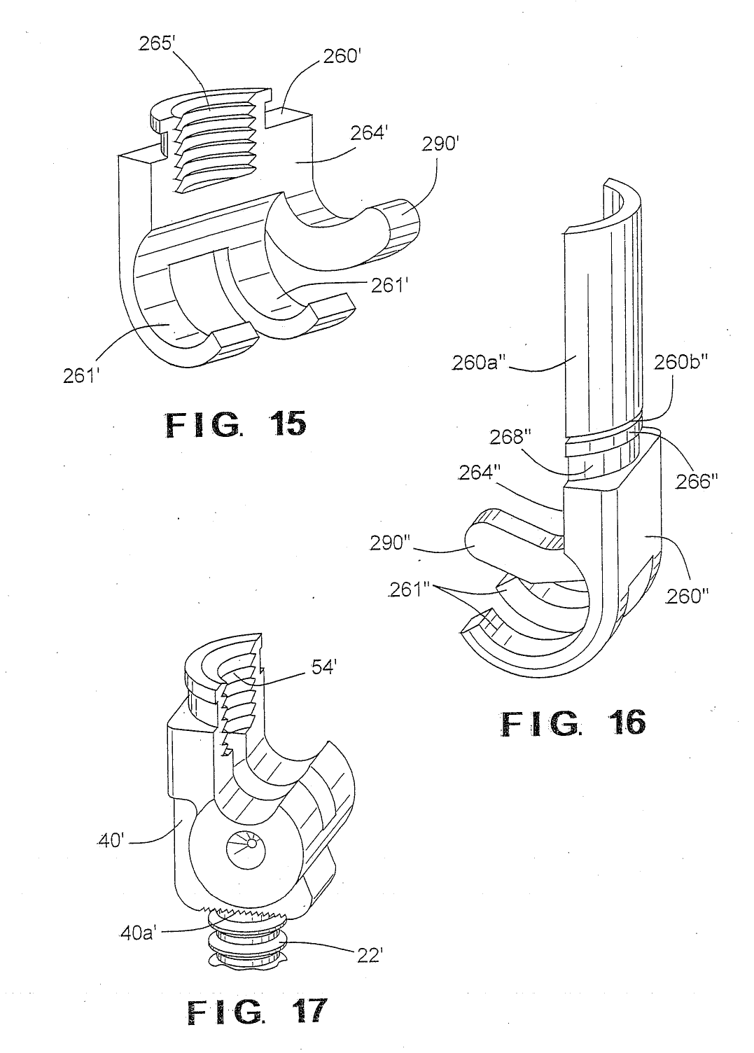

FIG. 15 is a perspective view of a modified pivot member 260′ of a seventh embodiment of a pedicle screw in accordance with this invention. The pivot member 260′ is identical to the pivot member 260 described above, except that the pivot fulcrum 290′ is located laterally outside of the mounting members 261′. The pivot fulcrum 290′ can, as described above, be provided at any desired location on the pivot member 290, both between and outside of the mounting members.

FIG. 16 is a perspective view of another modified pivot member 260″ of an eighth embodiment of a pedicle screw in accordance with this invention. The pivot member 260″ is identical to the pivot member 260 described above, except that the pivot member 260″ further includes an elongated extension 260a″. The illustrated extension 260a″ extends from the locking lip 266″ located at the end portion thereof and is provided to facilitate the installation and movement of the pivot member 260″ and to facilitate the insertion of the alignment rod 16. If desired, a reduced thickness region 260b″ may be provided between the extension 260a″ and the locking lip 266″ to facilitate the removal of the extension 260a″ following installation.

FIG. 17 is a perspective view of a modified stationary member 40′ of a ninth embodiment of a pedicle screw in accordance with this invention. The stationary member 40′ is identical to the stationary member 40 described above, except that the stationary member 40′ further includes a non-planar surface portion 40a′ that is provided on the bottom surface thereof adjacent to the upper end of the fastener portion 22. The non-planar surface portion 40a′ can be embodied as one or more serrations, spikes, or other structures that can engage the underlying bone of the vertebra 14 and improve the fixation of the pedicle screw 20 thereto. The non-planar surface portion 40a′ may alternatively (or additionally) be provided on the pivot member 60.

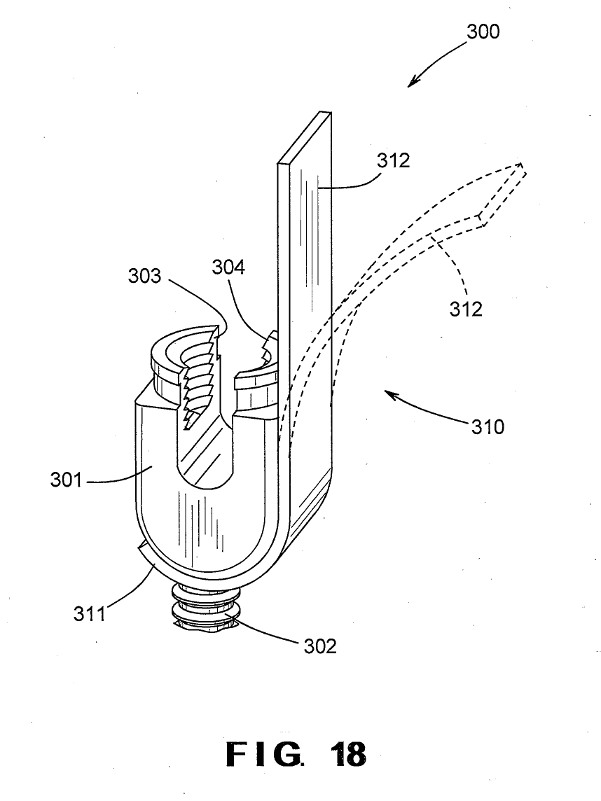

FIG. 18 is a perspective view of a tenth embodiment of a pedicle screw, indicated generally at 300, in accordance with this invention. The pedicle screw 300 includes a head portion 301 having a threaded shank portion 302 extending therefrom. The head portion 301 of the pedicle screw 300 includes a generally U-shaped yoke portion that is provided for receiving and securing the alignment rod 16 therein. Unlike the various embodiments described above, the generally U-shaped yoke portion is defined by first and second lateral surfaces 303 and 304 that are fixed in position relative to one another. The first and second lateral surfaces 303 and 304 have respective semi-annular, internally threaded sections 303a and 304a provided thereon for cooperation with the retention member 80 in the same manner as described above. The structure of the pedicle screw 300 thus far described is conventional in the art.

Unlike known pedicle screws, however, the tenth embodiment of the pedicle screw 300 includes a guide mechanism, indicated generally at 310, to facilitate the process of installing the alignment rod 16 within the generally U-shaped yoke portion of the head portion 301. In the illustrated embodiment, the guide mechanism 310 includes an attachment portion 311 and a guide portion 312. In the illustrated embodiment, the attachment portion 311 and the guide portion 312 are integrally formed from a single piece of material, although such is not required. The attachment portion 311 and the guide portion 312 may be formed from any desired material or group of materials.

The attachment portion 311 of the guide mechanism 310 is releasably connected to the head portion 301 of the pedicle screw 300. In the illustrated embodiment, the attachment portion 311 is generally U-shaped and is sized to frictionally engage the lower surface of the head portion 301 of the pedicle screw 300. However, the attachment portion 311 may be releasably secured to the head portion 301 of the pedicle screw 300 in any desired manner or, alternatively, may be permanently secured thereto.

The guide portion 312 of the guide mechanism 310 extends from the attachment portion 311. In the illustrated embodiment, the guide portion 312 extends generally parallel to the sides of the U-shaped yoke portion of the head portion 301, although such is not required. The guide portion 312 is preferably formed from a material that allows it to be flexed from a closed position (illustrated in solid lines) to an opened position (illustrated in dotted lines). The guide portion 312 can be moved to the opened position and used in the manner described above to facilitate the process of installing the alignment rod 16 within the generally U-shaped yoke portion of the head portion 301. Thereafter, the guide mechanism 310 can be removed from the pedicle screw 300. If the attachment portion 311 of the guide mechanism 310 is releasably connected to the head portion 301 of the pedicle screw 300, then the entire guide mechanism 310 can be removed from the head portion 301 of the pedicle screw 300. If, on the other hand, the attachment portion 311 of the guide mechanism 310 is permanently connected to the head portion 301 of the pedicle screw 300, then the guide portion 312 of the guide mechanism 310 can be removed from the attachment portion 311 thereof. To facilitate this, a groove (not shown) or other structure may be provided at the junction between the attachment portion 311 and the guide portion 312 of the guide mechanism 310.

The above detailed description of this invention is given for explanatory purposes. It will be apparent to those skilled in the art that numerous changes and modifications other than those cited can be made without departing from the scope of the invention. Accordingly, the whole of the foregoing description is to be construed in an illustrative and not a limitative sense, the scope of the invention being defined by the appended claims.

What is claimed is:

1. A pedicle screw for use in the treatment of a spinal deformity comprising:
a head assembly including a stationary member and a movable member that is supported on the stationary member for movement relative thereto between an opened position and a closed position, wherein the head assembly is adapted to receive an alignment rod therein for treatment of a spinal deformity
a fastener portion extending from the head assembly and adapted to be secured to a vertebra of a spinal column.

2. The pedicle screw defined in claim 1 wherein the head assembly is fixed in position relative to the fastener portion.

3. The pedicle screw defined in claim 1 wherein the head assembly is movable relative to the fastener portion.

4. The pedicle screw defined in claim 1 wherein the movable member is supported on the stationary member for pivoting movement relative thereto.

5. The pedicle screw defined in claim 1 wherein the stationary member has an engagement surface provided thereon, and wherein the movable member has a mounting member provided thereon that engages the engagement surface to support the pivot member on the stationary member for movement relative thereto.

6. The pedicle screw defined in claim 5 wherein the movable member has a plurality of mounting members provided thereon that each engage the engagement surface to support the pivot member on the stationary member for movement relative thereto.

7. The pedicle screw defined in claim 5 wherein the engagement surface and the mounting member are each generally cylindrically in shape.

8. The pedicle screw defined in claim 1 wherein the stationary member has a pivot stop provided thereon, and wherein the movable member has a pivot stop surface provided thereon that engages the pivot stop provided on the stationary member to prevent movement of the pivot member beyond the opened position.

9. The pedicle screw defined in claim 1 further including a locking member that retains the stationary member and the movable member in the closed position.

10. The pedicle screw defined in claim 1 wherein the stationary member and the movable member have respective locking grooves provided thereon, and wherein the locking member is disposed in the locking grooves to retain the stationary member and the movable member in the closed position.

11. The pedicle screw defined in claim 1 further including a fulcrum provided on the movable member that is adapted to be engaged by an alignment rod to automatically move the movable member from the opened position to the closed position relative to the stationary member.

12. The pedicle screw defined in claim 1 wherein at least one of the stationary member and the movable member has a non-planar surface provided thereon that is adapted to engage the vertebra and improve the fixation of the pedicle screw thereto.

13. The pedicle screw defined in claim 12 wherein the non-planar surface includes one or more of serrations and spikes.

14. A method for aligning a plurality of vertebrae in a spine comprising the steps of:
(a) providing a plurality of pedicle screws, wherein each pedicle screw includes a head assembly and a fastener portion extending from the head assembly, wherein the head assembly includes a stationary member and a movable member that is supported on the stationary member for movement relative thereto between an opened position and a closed position;
(b) securing each of the pedicle screws to respective vertebrae in a spine;
(c) orienting each of the head assemblies of the pedicle screws in the opened position;
(d) disposing an alignment rod in each of the head assemblies of the pedicle screws; and
(e) orienting each of the head assemblies of the pedicle screws in the closed position such that each of the plurality of vertebrae in the spine are aligned.

15. The method defined in claim 14 wherein step (a) is performed by providing a plurality of pedicle screws, wherein each of the movable members is supported on the respective stationary member for pivoting movement relative thereto.

16. The method defined in claim 14 wherein step (a) is performed by providing a plurality of pedicle screws, wherein each of the movable members includes a fulcrum, and wherein during step (d), the alignment rod engages the fulcrums to automatically move the movable members from the opened position to the closed positions relative to the stationary members.

17. The method defined in claim 14 including a further step (e) of retaining each of the stationary members and the movable members in the closed positions.

18. The method defined in claim 14 wherein step (e) is performed by installing a locking member that retains each of the stationary members and the movable members in the closed positions.
